(19) United States
(12) Patent Application Publication
(10) Pub. No.: US 2025/0017968 A1
(43) Pub. Date: Jan. 16, 2025

(54) ANTIBODY SPECIFIC TO EPHRIN TYPE-A RECEPTOR 10, FUSION PROTEIN CONTAINING THE SAME, CHIMERIC ANTIGEN RECEPTOR T-CELL EXPRESSING THE SAME AND USES THEREOF

(71) Applicant: CHINA MEDICAL UNIVERSITY, Taichung (TW)

(72) Inventors: Long-Bin JENG, Taichung (TW); Shih-Ping LIU, Taichung (TW); Chie-Hong WANG, Taichung (TW)

(21) Appl. No.: 18/712,200

(22) PCT Filed: Nov. 22, 2022
(86) PCT No.: PCT/CN2022/133444

§ 371 Date: May 21, 2024

Related U.S. Application Data
(63) Continuation of application No. 63/264,378, filed on Nov. 22, 2021.

(51) Int. Cl. A61K 35/17; A61K 39/00; A61K 47/68; A61P 35/00; C07K 16/28; G01N 33/573; G01N 33/68

(52) U.S. Cl. CPC A61K 35/17; A61K 39/4611; A61K 39/4631; A61K 39/464422; A61K 47/6849; A61P 35/00; C07K 16/2866; G01N 33/573; G01N 33/6872; A61K 2239/13; A61K 2239/17; A61K 2239/29

(57) ABSTRACT

The present disclosure relates to an antibody or antigen-binding fragment thereof that is specific to ephrin type-A receptor 10 (EphA10) and a fusion protein containing the antibody or antigen-binding fragment thereof. The present disclosure also relates to a pharmaceutical composition, a method for treating and/or preventing diseases and/or disorders caused by EphA10 in a subject in need, and a method for detecting EphA10 in a sample.

### PRIORITY INFORMATION

This application is a 371 National Phase Application of International Patent Application No. PCT/CN2022/133444, filed Nov. 22, 2022, which claims benefit of and priority to U.S. Provisional Patent Application No. 63/264,378, filed Nov. 22, 2021, the contents of which are incorporated by reference in their entirety.

### SEQUENCE LISTING

The instant application contains a Sequence Listing which is submitted electronically in .xml format and is hereby incorporated by reference in its entirety. The .xml copy, created on May 21, 2024, is named “G4590-14900NP_20240521_SeqList.xml” and is 11 kilobytes in size.

### FIELD OF THE DISCLOSURE

The present disclosure relates to an antibody or antigen-binding fragment thereof, which is specific to ephrin type-A receptor 10 (EphA10), a fusion protein containing the same, a chimeric antigen receptor T-cell expressing the same and uses thereof.

### BACKGROUND OF THE DISCLOSURE

The utilization of therapeutic monoclonal antibodies for the treatment of human diseases including solid and hematological malignancies is well validated and established. Particularly useful for therapeutic applications are chimeric or humanized monoclonal antibodies due to their reduced immunological side effects. Among various immunotherapies developed for cancer treatments, chimeric antigen receptor (CAR) T-cell therapy is regarded as a powerful strategy. Generally, CAR T-cell therapy is performed by genetic modification of patient's T cells to express a tumor-specific CAR, ex vivo cell expansion and re-infusion back to the patient. Although an up to 92% full recovery rate is achieved in clinical trials in acute lymphocytic leukemia, the clinical experience of CAR T-cell therapy is still limited by several challenges, e.g., the expansion of CAR-T cells and the selection of tumor-specific antigen.

Thus, there is need for developing a novel approach to treating cancer.

### SUMMARY OF THE DISCLOSURE

The present disclosure provides a novel cancer antigen receptor and uses thereof.

Accordingly, the present disclosure provides an antibody or antigen-binding fragment thereof that is specific to an epitope of EphA10. The antibody according to the disclosure is thus useful for treating and/or preventing diseases and/or disorders caused by caused by or related to EphA10 activity and/or signaling. The antibody of the disclosure is also useful for detecting EphA10.

In some embodiments, the disclosure provides an antibody or antigen-binding fragment thereof that is specific to an epitope in EphA10; wherein the antibody or antigen-binding fragment thereof comprises complementarity determining regions (CDRs) of a heavy chain variable region and complementarity determining regions of a light chain variable region, wherein the complementarity determining regions of the heavy chain variable region comprise CDRH1, CDRH2 and CDRH3 regions, and the complementarity determining regions of the light chain variable region comprise CDRL1, CDRL2 and CDRL3 regions, and wherein:
the CDRH1 region comprises the amino acid sequence of SEQ ID NO: 1 or a substantially similar sequence thereof; the CDRH2 region comprises the amino acid sequence of SEQ ID NO: 2 or a substantially similar sequence thereof; the CDRH3 region comprises the amino acid of SEQ ID NO: 3 or a substantially similar sequence thereof; and
the CDRL1 region comprises the amino acid sequence of SEQ ID NO: 4 or a substantially similar sequence thereof; the CDRL2 region comprises the amino acid sequence of SEQ ID NO: 5 or a substantially similar sequence thereof; the CDRL3 region comprises the amino acid sequence of SEQ ID NO: 6 or a substantially similar sequence thereof.

In some embodiments, the antibody or antigen-binding fragment thereof comprises a heavy chain variable region comprising the amino acid sequence of SEQ ID NO: 7 or a substantially similar sequence thereof; and a light chain variable region comprising the amino acid sequence of SEQ ID NO: 8 or a substantially similar sequence thereof.

In some embodiments, the antibody is a monoclonal antibody, chimeric antibody, humanized antibody, human antibody, or nanobody.

In some embodiments, the antibody is multi-specific.

In some embodiments of the disclosure, the antibody or antigen-binding fragment thereof is conjugated with a therapeutic agent. Examples of the therapeutic agent include but are not limited to antimetabolites, alkylating agents, alkylating-like agents, DNA minor groove alkylating agents, anthracyclines, antibiotics, calicheamicins, antimitotic agents, topoisomerase inhibitors, HDAC inhibitor, proteasome inhibitors, and radioisotopes.

In some embodiments, the present disclosure also provides to a fusion protein comprising an antigen binding portion comprising the antibody or antigen-binding fragment thereof as disclosed herein; and 30 at least one costimulatory signaling domain.

Examples of the costimulatory signaling domain include, but are not limited to CD27, CD28, 4-1BB, OX40, CD30, CD40, ICOS, ICAM-1, LFA-1, CD2, CD7, LIGHT, NKG2C, B7-H3, a ligand that specifically binds with CD83, CDS, GITR, BAFFR, HVEM, SLAMf7, NKP80, CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, C49f, ITGAD, CD11d, ITGAE, CD103 ITGAL, CD11a, LFA-1, ITGAM, CD11b, ITGAX, CD11c, ITGB1, CD29, ITGB2, CD18, ITGB7, TNFR2, TRANCE/RANKL, DNAM1, SLAMF4, CD84, CD96, CEACAM1, CRTAM, Ly9, PSGL1, C100, CD69, SLAMF6, SLAM, BLAME, SELPLG, LTBR, LAT, GADS, PAG/Cbp, SLP-76, NKp44, NKp30, or NKp46. In some embodiments, the costimulatory signaling domain is CD28 or 4-1BB.

In some embodiments of the disclosure, the fusion protein further comprises a primary signaling domain.

In some embodiments of the disclosure, the fusion protein further comprises a hinge domain.

In some embodiments of the disclosure, the fusion protein comprises the antigen binding portion, a CD8 hinge, CD28, 41-BB and CD3ζ.

In some embodiments of the disclosure, the antibody or antigen-binding fragment thereof or the fusion protein is expressed on a surface of a cell. The cell may be an immune cell or a stem cell. In one embodiment of the disclosure, the immune cell is a T cell. In another aspect, the stem cell may be an induced pluripotent stem cell.

The present disclosure also provides a vector encoding the antibody or antigen-binding fragment thereof or the fusion protein.

In another aspect, the present disclosure provides a genetically engineered cell expressing the antibody or antigen-binding fragment thereof or the fusion protein, or containing the vector. The genetically engineered cell may be an immune cell or a stem cell. Also, the present disclosure provides an immune cell, which is differentiated from the genetically engineered cell.

The present disclosure provides a pharmaceutical composition comprising an effective amount of the antibody or antigen-binding fragment thereof, the fusion protein, the genetically engineered cell or the immune cell.

The present disclosure provides a method for inhibiting EphA 10-mediated signaling in a subject in need, comprising administering to the subject the pharmaceutical composition. Alternatively, the present disclosure provides a pharmaceutical composition for use in inhibiting EphA10-mediated signaling in a subject in need, comprising an effective amount of the antibody or antigen-binding fragment thereof, the fusion protein, the genetically engineered cell or the immune cell as disclosed herein.

The present disclosure also provides a method for treating, prophylactic treating and/or preventing diseases and/or disorders caused by or related to EphA10 activity and/or signaling in a subject afflicted with the diseases and/or disorders, comprising administering to the subject the pharmaceutical composition. Alternatively, the present disclosure provides a pharmaceutical composition for use in treating, prophylactic treating and/or preventing diseases and/or disorders caused by or related to EphA10 activity and/or signaling in a subject afflicted with the diseases and/or disorders, comprising an effective amount of the antibody or antigen-binding fragment thereof, the fusion protein, the genetically engineered cell or the immune cell as disclosed herein.

The present disclosure still also provides a method for treating, prophylactic treating and/or preventing tumor in a subject afflicted with the tumor, comprising administering to the subject the pharmaceutical composition. In some embodiments of the disclosure, the tumor is a solid tumor. Examples of the tumor include but are not limited to renal cell carcinoma, pancreatic carcinoma, breast cancer, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, or melanoma. Alternatively, the present disclosure provides a pharmaceutical composition for use in treating, prophylactic treating and/or preventing tumor in a subject afflicted with the tumor, comprising an effective amount of the antibody or antigen-binding fragment thereof, the fusion protein, the genetically engineered cell or the immune cell as disclosed herein.

The present disclosure provides a method for detecting EphA10 in a sample comprising contacting the sample with the antibody or antigen-binding fragment thereof.

The present disclosure provides a method for neutralizing EphA10 in a subject in need, comprises administering to the subject the antibody or antigen-binding fragment thereof. Alternatively, the present disclosure provides a pharmaceutical composition for use in neutralizing EphA10 in a subject in need, comprising an effective amount of the antibody or antigen-binding fragment thereof, the fusion protein, the genetically engineered cell or the immune cell as disclosed herein.

The present disclosure provides a kit for detecting EphA10 in a sample, wherein the kit comprises the antibody or antigen-binding fragment thereof or the fusion protein.

The present disclosure is described in detail in the following sections. Other characteristics, purposes and advantages of the present disclosure can be found in the detailed description and claims.

### BRIEF DESCRIPTION OF THE DRAWINGS

EphAEphA FIG. 1 shows the results of binding specificity assay of the anti-EphA10 antibody CMU #13.

FIG. 2 shows the fragment of the CAR-T vector containing EphA10 (CMU #13)-specific ScFv, CD8 hinge, CD28 TM and IC, 41-BB and CD33.

FIG. 3 shows that the increased cytotoxic effect of anti-EphA10 CAR-T cells was validated by co-culturing the anti-EphA10 CAR T cells with breast cancer cells.

FIG. 4 shows the results of the tumor inhibition effect of anti-EphA10-CAR-T cell in a breast cancer xenograft animal model.

FIG. 5 shows that Anti-EphA10 CAR-T cell triggers massive apoptosis in tumors. Scale=1000 μm.

### DETAILED DESCRIPTION OF THE DISCLOSURE

It is understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise.

The term “antibody”, as used herein, means any antigen-binding molecule or molecular complex comprising at least one complementarity determining region (CDR) that specifically binds to or interacts with a particular antigen (EphA10). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-EphA10 antibody (or antigen-binding portion thereof) may be identical to the human germline sequences, or may be naturally or artificially modified. An amino acid consensus sequence may be defined based on a side-by-side analysis of two or more CDRs.

As used herein, the term “being specific to” means that an antibody does not cross react to a significant extent with other epitopes.

As used herein, the term “epitope” refers to the site on the antigen to which an antibody binds.

As used herein, the term “complementarity determining region” (CDR) refers to the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

The term “monoclonal antibody” as used herein is not limited to antibodies produced through hybridoma technology. A monoclonal antibody is derived from a single clone, including any eukaryotic, prokaryotic, or phage clone, by any means available or known in the art.

The term “chimeric” antibody as used herein refers to an antibody having variable sequences derived from a non-human immunoglobulin and human immunoglobulin constant regions, typically chosen from a human immunoglobulin template.

“Humanized” forms of non-human antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence.

As used herein, the term “nanobody” refers to an antibody comprising the small single variable domain (VHH of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex.

As used herein, the term “a fusion protein,” also known as “a chimeric protein,” refers to a protein that is translated from the joining of two or more genes originally coded for separate proteins.

“Costimulatory signaling domain,” as that term is used herein, refers to a molecule, e.g., an endogenous molecule, of the CAR-T cell that, upon binding to its cognate counter ligand on a target cell, enhance, e.g., increases, an immune effector response.

As used in the present disclosure, the term “therapeutic agent” means any compound, substance, drug, drug or active ingredient having a therapeutic or pharmacological effect that is suitable for administration to a mammal, for example a human. As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “T cell” includes CD4⁺ T cells and CD8⁺ T cells. The term T cell also includes T helper 1 type T cells, T helper 2 type T cells, T helper 17 type T cells and inhibitory T cells.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

The term “pluripotent” when used in reference to a “pluripotent cell” refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse teratoma formation assay. Pluripotency is also evidenced by the expression of embryonic stem (ES) cell markers, although a preferred test for pluripotency is the demonstration of the capacity to differentiate into cells of each of the three germ layers.

As used herein, the terms “iPS cell” and “induced pluripotent stem cell” are used interchangeably and refers to a pluripotent cell technically derived (e.g., induced by complete or partial reversal) from a differentiated cell (e.g. a non-pluripotent cell), typically an adult differentiated cell, for example, by contacting the cell with at least one compound of any compounds selected from xanthine, xanthosine, hypoxanthine, or analogs thereof, e.g. compounds with a xanthine nucleus.

As used herein, the term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “genetically engineered” or “genetic engineering” of cells means manipulating genes using genetic materials for the change of gene copies and/or gene expression level in the cell. The genetic materials can be in the form of DNA or RNA. The genetic materials can be transferred into cells by various means including viral transduction and non-viral transfection. After being genetically engineered, the expression level of certain genes in the cells can be altered permanently or temporarily.

In the context of cell ontogeny, the adjective “differentiated” or “differentiating” is a relative term. A “differentiated cell” is a cell that has progressed further down the developmental pathway than the cell it is being compared with. Thus, stem cells can differentiate to lineage-restricted precursor cells, which in turn can differentiate into other types of precursor cells further down the pathway, and then to an end-stage differentiated cell, which plays a characteristic role in a certain tissue type, and may or may not retain the capacity to proliferate further.

As used in the present invention, the term “pharmaceutical composition” means a mixture containing a therapeutic agent administered to a mammal, for example a human, for preventing, treating, or eliminating a particular disease or pathological condition that the mammal suffers.

As used herein, the term “therapeutically effective amount” or “efficacious amount” refers to the amount of an antibody that, when administered to a mammal or other subject for treating a disease, is sufficient to effect such treatment for the disease.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

“Cancer,” “tumor,” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3rd ed. 1994)). Although transformation can arise from infection with a transforming virus and incorporation of new genomic DNA, or uptake of exogenous DNA, it can also arise spontaneously or following exposure to a carcinogen.

As used herein, the term “sample” encompasses a variety of sample types obtained from an individual, subject or patient and can be used in a diagnostic or monitoring assay. The definition encompasses blood and other liquid samples of biological origin, solid tissue samples such as a biopsy specimen or tissue cultures or cells derived therefrom and the progeny thereof.

The present disclosure develops an antibody or antigen-binding fragment thereof that is specific to an epitope in ephrin type-A receptor 10.

EphA10 is a member of ephrin receptors, the largest subfamily of receptor tyrosine kinases (RTKs), and has an important function in development, angiogenesis, and cell differentiation. Previous studies indicated the therapeutic application of developing monoclonal antibody targeting EphA10 for cancer immunotherapy. It is found that (1) EphA10 levels are very low in normal tissues except the testis; (2) EphA10 levels are higher in various types of cancer than in normal tissues; (3) EphA10 deletion induced tumor regression by enhancing CTL-mediated antitumor immunity.

Particularly, the antibody or antigen-binding fragment thereof comprises complementarity determining regions (CDRs) of a heavy chain variable region and complementarity determining regions of a light chain variable region, wherein the complementarity determining regions of the heavy chain variable region comprise CDRH1, CDRH2 and CDRH3 regions, and the complementarity determining regions of the light chain variable region comprise CDRL1, CDRL2 and CDRL3 regions, and wherein:
the CDRH1 region comprises the amino acid sequence of SEQ ID NO: 1 or a substantially similar sequence having at least 90%, at least 95%, at least 98% or at least 99% sequence identity; the CDRH2 region comprises the amino acid sequence of SEQ ID NO: 2 or a substantially similar sequence having at least 90%, at least 95%, at least 98% or at least 99% sequence identity; the CDRH3 region comprises the amino acid sequence of SEQ ID NO: 3 or a substantially similar sequence having at least 90%, at least 95%, at least 98% or at least 99% sequence identity; and
the CDRL1 region comprises the amino acid sequence of SEQ ID NO: 4 or a substantially similar sequence having at least 90%, at least 95%, at least 98% or at least 99% sequence identity; the CDRL2 region comprises the amino acid sequence of SEQ ID NO: 5 or a substantially similar sequence having at least 90%, at least 95%, at least 98% or at least 99% sequence identity; the CDRL3 region comprises the amino acid sequence of SEQ ID NO: 6 or a substantially similar sequence having at least 90%, at least 95%, at least 98% or at least 99% sequence identity.

The sequence listing is shown in Table 1.

TABLE 1

| SEQ | | |
|---|---|---|
| ID | Sequence | |
| NO | name | sequence |
| 1 | VH CDR1 | SYAIS |
| 2 | VH CDR2 | GIIPIFGTANYAQKFQG |
| 3 | VH CDR3 | EGDSYGYYYGMDV |
| 4 | VL CDR1 | SGSSSNIGSNYVY |
| 5 | VL CDR2 | ENYLRPS |
| 6 | VL CDR3 | AAWDDSLNGYV |
| 7 | VH | QVQLVQSGAEVKKPGSSVKVSCKASGGTFSSYAISWVRQAPG |
| | | QGLEWMGGIIPIFGTANYAQKFQGRVTITADESTSTAYMELSSL |
| | | RSEDTAVYYCAREGDSYGYYYGMDVWGQGTTVTVSS |
| 8 | VL | QPVLTQPPSASGTPGQRVTISCSGSSSNIGSNYVYWYQQLPGTA |
| | | PKLLIYENYLRPSGVPDRFSGSKTGTSASLAISGLQSEDEADYY |
| | | CAAWDDSLNGYVFGTGTQLTVLS |
| 9 | scFv | QVQLVQSGAEVKKPGSSVKVSCKASGGTFSSYAISWVRQAPG |
| | amino acid | QGLEWMGGIIPIFGTANYAQKFQGRVTITADESTSTAYMELSSL |
| | | RSEDTAVYYCAREGDSYGYYYGMDVWGQGTTVTVSSGGGGS |
| | | GGGGSGGGGSQPVLTQPPSASGTPGQRVTISCSGSSSNIGSNYV |
| | | YWYQQLPGTAPKLLIYENYLRPSGVPDRFSGSKTGTSASLAISG |
| | | LQSEDEADYYCAAWDDSLNGYVFGTGTQLTVLS |
| 10 | scFv DNA | CAGGTCCAGCTGGTGCAGTCTGGGGCTGAGGTGAAGAAGCC |
| | | TGGGTCCTCGGTGAAGGTCTCCTGCAAGGCTTCTGGAGGCA |
| | | CCTTCAGCAGCTATGCTATCAGCTGGGTGCGACAGGCCCCTG |
| | | GACAAGGGCTTGAGTGGATGGGAGGGATCATCCCTATCTTT |
| | | GGTACAGCAAACTACGCACAGAAGTTCCAGGGCAGAGTCAC |
| | | GATTACCGCGGACGAATCCACGAGCACAGCCTACATGGAGC |
| | | TGAGCAGCCTGAGATCTGAGGACACGGCCGTGTATTACTGT |
| | | GCGAGAGAAGGGGACAGCTATGGTTACTACTACGGTATGGA |
| | | CGTCTGGGGGCAGGGGACCACGGTCACCGTCTCGAGTGGTG |
| | | GAGGCGGTTCAGGCGGAGGTGGTTCTGGCGGTGGCGGATCG |
| | | CAGCCTGTGCTGACTCAGCCCCCCTCAGCGTCTGGGACCCCC |
| | | GGGCAGAGGGTCACCATCTCTTGTTCTGGAAGCAGCTCCAA |
| | | CATCGGAAGTAATTATGTATACTGGTACCAGCAGCTCCCAG |
| | | GAACGGCTCCCAAACTCCTCATCTATGAGAATTATCTGCGGC |
| | | CCTCAGGGGTCCCTGACCGATTCTCTGGCTCCAAGACTGGCA |
| | | CCTCAGCCTCCCTGGCCATCAGTGGGCTCCAGTCTGAGGATG |
| | | AGGCTGATTATTACTGTGCAGCATGGGATGACAGCCTGAAT |
| | | GGTTATGTCTTCGGAACTGGGACCCAGCTCACCGTTTTAAGT |

In some embodiments, the antibody or antigen-binding fragment thereof comprises a heavy chain variable region comprising the amino acid sequence of SEQ ID NO: 7 or a substantially similar sequence thereof; and a light chain variable region comprising the amino acid sequence of SEQ ID NO: 8 or a substantially similar sequence thereof. In some further embodiments, the antibody or antigen-binding fragment thereof comprises a heavy chain variable region comprising the amino acid sequence of SEQ ID NO: 7 or a substantially similar sequence thereof having at least 90%, at least 95%, at least 98% or at least 99% sequence identity. In some further embodiments, the antibody or antigen-binding fragment thereof comprises a light chain variable region comprising the amino acid sequence of SEQ ID NO: 8 or a substantially similar sequence thereof having at least 90%, at least 95%, at least 98% or at least 99% sequence identity.

The antibody according to the disclosure can be full-length (for example, an IgG1 or IgG4 antibody) or may comprise only an antigen-binding portion (for example, a Fab, F(ab′)₂ or scFv fragment), and may be modified to affect functionality as needed.

Various techniques known to persons of ordinary skill in the art can be used to determine whether an antibody “is specific to one or more amino acids” within a polypeptide or protein. Exemplary techniques include, e.g., routine cross-blocking assay such as that described Antibodies, Harlow and Lane (Cold Spring Harbor Press, Cold Spring Harb., N.Y.), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody specifically binds is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267 (2): 252-259; Engen and Smith (2001) Anal. Chem. 73:256A-265A.

One can easily determine whether an antibody is specific to the same epitope as, or competes for binding with, a reference anti-EphA10 antibody by using routine methods known in the art. For example, to determine if a test antibody binds to the same epitope as a reference anti-EphA 10 antibody of the disclosure, the reference antibody is allowed to bind to an EphA 10 protein. Next, the ability of a test antibody to bind to the EphA 10 molecule is assessed. If the test antibody is able to bind to EphA10 following saturation binding with the reference anti-EphA10 antibody, it can be concluded that the test antibody binds to a different epitope than the reference anti-EphA10 antibody. On the other hand, if the test antibody is not able to bind to the EphA10 molecule following saturation binding with the reference anti-EphA10 antibody, then the test antibody may bind to the same epitope as the epitope bound by the reference anti-EphA10 antibody of the disclosure. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay. Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

The antibody also includes an antigen-binding fragment of a full antibody molecule. An antigen-binding fragment of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of an antigen-binding fragment includes: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody typically comprises at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (V) V_{H}-C_{H1}-C_{H2}-C_{H3}, (vi) V_{H}-C_{H2}-C_{H3}; (vii) V_{H}-C_{L}; (viii) V_{L}-C_{H1}; (ix) V_{L}-C_{H2}; (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The anti-EphA10 antibody disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes an antibody, and an antigen-binding fragment thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another mammalian germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes an anti-EphA10 antibody comprising variants of any of the V_{H}, V_{L}, and/or CDR amino acid sequences disclosed herein having one or more conservative substitutions. For example, the present disclosure includes an anti-EphA10 antibody having V_{H}, V_{L}, and/or CDR amino acid sequences with, e.g., 10 or fewer, 8 or fewer, 6 or fewer, 4 or fewer, etc. conservative amino acid substitutions relative to any of the V_{H}, V_{L}, and/or CDR amino acid sequences disclosed herein.

In some embodiments of the disclosure, the antibody according to the disclosure is a humanized antibody. In order to improve the binding affinity of the humanized antibody according to the disclosure, some amino acid residues in the human framework region are replaced by the corresponding amino acid residues in the species of CDRs; e.g. a rodent.

The antibodies of the present disclosure may be monospecific, bi-specific, or multispecific. Multispecific antibodies may be specific for different epitopes of one target polypeptide or may contain antigen-binding domains specific for more than one target polypeptide. The anti-EphA10 antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present disclosure includes bi-specific antibodies wherein one arm of an immunoglobulin is specific for EphA10 or a fragment thereof, and the other arm of the immunoglobulin is specific for a second therapeutic target or is conjugated to a therapeutic moiety.

In one embodiment of the disclosure, the antibody or antigen-binding fragment thereof is conjugated with a therapeutic agent.

In some embodiments of the disclosure, the therapeutic agent represents a cytostatic or cytotoxic agent or an isotope-chelating agent with corresponding radioisotopes. Examples of the cytostatic or cytotoxic agent include, without limitation, antimetabolites (e.g., fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, or pemetrexed); alkylating agents (e.g., cmelphalan, chlorambucil, busulfan, thiotepa, ifosfamide, carmustine, lomustine, semustine, streptozocin, dacarbazine, mitomycin C, cyclophosphamide, mechlorethamine, uramustine, dibromomannitol, tetranitrate, procarbazine, altretamine, mitozolomide, or temozolomide); alkylating-like agents (e.g., cisplatin, carboplatin, nedaplatin, oxaliplatin, satraplatin, or triplatin); DNA minor groove alkylating agents (e.g., duocarmycins such as CC-1065, and any analogs or derivatives thereof; pyrrolobenzodiazapenes, or any analogs or derivatives thereof); anthracyclines (e.g., daunorubicin, doxorubicin, epirubicin, idarubicin, or valrubicin); antibiotics (e.g., dactinomycin, bleomycin, mithramycin, anthramycin, streptozotocin, gramicidin D, mitomycins (e.g., mitomycin C); calicheamicins; antimitotic agents (including, e.g., maytansinoids (such as DM1, DM3, and DM4), auristatins (including, e.g., monomethyl auristatin E (MMAE) and monomethyl auristatin F (MMAF)), dolastatins, cryptophycins, vinca alkaloids (e.g., vincristine, vinblastine, vindesine, vinorelbine), taxanes (e.g., paclitaxel, docetaxel, or a novel taxane), tubulysins, and colchicines); topoisomerase inhibitors (e.g., irinotecan, topotecan, camptothecin, etoposide, teniposide, amsacrine, or mitoxantrone); HDAC inhibitor (e.g., vorinostat, romidepsin, chidamide, panobinostat, or belinostat); proteasome inhibitors (e.g., peptidyl boronic acids); as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Examples of the isotope-chelating agents include, without limitation, ethylenediaminetetraacetic acid (EDTA), diethylenetriamine-N,N,N′,N″,N″-pentaacetate (DTPA), 1,4,7,10-tetraazacyclododecane-N,N′,N″,N′″-tetraacetate (DOTA), 1,4,7,10-tetrakis(2-hydroxypropyl)-1,4,7,10-tetraazacyclododecane (THP), triethylenetetraamine-N,N,N′,N″,N′″,N′″-hexaacetate (TTHA), 1,4,7,10-tetraazacyclododecane-N,N′,N″,N′-tetrakis(methylenephosphonate) (DOTP), and mercaptoacetyltriglycine (MAG3).

In some embodiments, the present disclosure also provides to a fusion protein comprising an antigen binding portion comprising the antibody or antigen-binding fragment thereof as disclosed herein; and

at least one costimulatory signaling domain.

In an embodiment, a costimulatory signaling domain produces an intracellular signal when an extracellular domain, e.g., an antigen binding domain to which it is fused, or coupled by a dimerization switch, binds cognate ligand. It is derived from a costimulatory molecule. It comprises sufficient costimulatory molecule sequence to produce an intracellular signal, e.g., when an extracellular domain, e.g., an antigen binding domain, to which it is fused, or coupled by a dimerization switch, binds cognate ligand. Examples of the costimulatory signaling domain include, but are not limited to CD27, CD28, 4-1BB, OX40, CD30, CD40, ICOS, ICAM-1, LFA-1, CD2, CD7, LIGHT, NKG2C, B7-H3, a ligand that specifically binds with CD83, CDS, GITR, BAFFR, HVEM, SLAMf7, NKP80, CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, C49f, ITGAD, CD11d, ITGAE, CD103 ITGAL, CD11a, LFA-1, ITGAM, CD11b, ITGAX, CD11c, ITGB1, CD29, ITGB2, CD18, ITGB7, TNFR2, TRANCE/RANKL, DNAM1, SLAMF4, CD84, CD96, CEACAM1, CRTAM, Ly9, PSGL1, C100, CD69, SLAMF6, SLAM, BLAME, SELPLG, LTBR, LAT, GADS, PAG/Cbp, SLP-76, NKp44, NKp30, or NKp46. In some embodiments, the costimulatory signaling domain is CD28 or 4-1BB.

In some embodiments of the disclosure, the fusion protein further comprises a primary signaling domain. A primary signaling domain that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 ζ, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In some embodiments, the primary signaling domain is CD3-ζ.

In some embodiments of the disclosure, the fusion protein further comprises a hinge domain. The antigen binding portion is generally followed by one or more “hinge domains,” which plays a role in positioning the antigen binding portion away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

In some embodiments of the disclosure, the fusion protein comprises the antigen binding portion, a CD8 hinge, CD28, 41-BB and CD33ζ.

In one embodiment of the disclosure, the antibody or antigen-binding fragment thereof or the fusion protein is expressed on the surface of a cell. Particularly, the cell is a T-cell or a stem cell, such as an iPSC. Induced pluripotent stem cells can be reprogrammed by inducing Yamanaka factors into somatic cells. Being like embryonic stem cells, iPSCs have the ability to be differentiated into cells of three-germ layers without the concern of ethic issue. With this property, iPSCs exhibit promising applications for clinical use.

In some embodiments of the disclosure, the antibody or antigen-binding fragment thereof or the fusion protein is in a form of chimeric antigen receptor.

The term “chimeric antigen receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. The generation and construction of CAR are generalized by Jayaraman et al., EBioMedicine 58 (2020) 102931; Zhang et al., Biomarker Research (2017) 5:22; Feins et al., Am J Hematol. (2019) 94:S3-S9; and Roselli et al., J Clin Invest. 2021; 131 (2):e142030.

The antibody or antigen-binding fragment thereof or the fusion protein may be encoded in a vector encoding the antibody or antigen-binding fragment thereof. An exemplary vector is a lentivirus vector. “Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

In another aspect, the present disclosure provides a genetically engineered cell expressing the antibody or antigen-binding fragment thereof or the fusion protein or containing the vector. The genetically engineered cell may be an immune cell or a stem cell. Also, the present disclosure provides an immune cell, which is differentiated from the genetically engineered cell.

In one embodiment of the disclosure, anti-EphA10 iPSCs and T cells with lentivirus carrying EphA10 gene are generated. Furthermore, the iPSCs are differentiated these into CAR-immune cells. The cytotoxic effects of these CAR-immune cells on cancer cells are observed. The present disclosure provides an approach to overcome the specificity of tumor antigen and produce limitless CAR-immune cells for clinical cancer treatment application.

The disclosure provides pharmaceutical compositions comprising the antibody or antigen-binding fragment thereof or the fusion protein, genetically engineered cell or immune cell of the present disclosure. The pharmaceutical compositions of the disclosure are formulated with suitable diluents, carriers, excipients, and other agents that provide improved transfer, delivery, tolerance, and the like. The compositions may be formulated for specific uses, such as for veterinary uses or pharmaceutical uses in humans. The form of the composition and the excipients, diluents and/or carriers used will depend upon the intended uses of the antibody and, for therapeutic uses, the mode of administration. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. When an antibody of the present disclosure is used for treating a condition or disease associated with EphA10 in an adult patient, it may be advantageous to intravenously administer the antibody of the present disclosure. Depending on the severity of the condition, the frequency and the duration of the treatment can be adjusted. Effective dosages and schedules for administering the antibody may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The present disclosure provides a method for inhibiting EphA10-mediated signaling in a subject in need, comprising administering to the subject the pharmaceutical composition. Alternatively, the present disclosure provides a pharmaceutical composition for use in inhibiting EphA 10-mediated signaling in a subject in need, comprising an effective amount of the antibody or antigen-binding fragment thereof, the fusion protein, the genetically engineered cell or the immune cell as disclosed herein.

The present disclosure also provides a method for treating, prophylactic treating and/or preventing diseases and/or disorders caused by or related to EphA10 activity and/or signaling in a subject afflicted with the diseases and/or disorders, comprising administering to the subject the pharmaceutical composition. Alternatively, the present disclosure provides a pharmaceutical composition for use in treating, prophylactic treating and/or preventing diseases and/or disorders caused by or related to EphA10 activity and/or signaling in a subject afflicted with the diseases and/or disorders, comprising an effective amount of the antibody or antigen-binding fragment thereof, the fusion protein, the genetically engineered cell or the immune cell as disclosed herein.

The present disclosure still also provides a method for treating, prophylactic treating and/or preventing tumor in a subject afflicted with the tumor, comprising administering to the subject the pharmaceutical composition. In some embodiments of the disclosure, the tumor is a solid tumor. Examples of the tumor include but are not limited to renal cell carcinoma, pancreatic carcinoma, breast cancer, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, or melanoma. Alternatively, the present disclosure provides a pharmaceutical composition for use in treating, prophylactic treating and/or preventing tumor in a subject afflicted with the tumor, comprising an effective amount of the antibody or antigen-binding fragment thereof, the fusion protein, the genetically engineered cell or the immune cell as disclosed herein.

The present disclosure provides a method for detecting EphA10 in a sample comprising contacting the sample with the antibody or antigen-binding fragment thereof.

The present disclosure provides a method for neutralizing EphA10 in a subject in need, comprises administering to the subject the antibody or antigen-binding fragment thereof. Alternatively, the present disclosure provides a pharmaceutical composition for use in neutralizing EphA10 in a subject in need, comprising an effective amount of the antibody or antigen-binding fragment thereof, the fusion protein, the genetically engineered cell or the immune cell as disclosed herein.

The present disclosure provides a kit for detecting EphA10 in a sample, wherein the kit comprises the antibody or antigen-binding fragment thereof or the fusion protein.

The anti-EphA10 antibody of the present disclosure may also be used to detect and/or measure EphA10, or EphA10-expressing cells in a sample, e.g., for diagnostic purposes. For example, an anti-EphA10 antibody, or fragment thereof, may be used to diagnose a condition or disease characterized by aberrant expression (e.g., over-expression, under-expression, lack of expression, etc.) of EphA10. Exemplary diagnostic assays for EphA10 may comprise, e.g., contacting a sample, obtained from a patient, with an anti-EphA10 antibody of the disclosure, wherein the anti-EphA10 antibody is labeled with a detectable label or reporter molecule. Alternatively, an unlabeled anti-EphA10 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure EphA 10 in a sample include enzyme-linked immunosorbent assay (ELISA), radioimmunoassay (RIA), and fluorescence-activated cell sorting (FACS).

The following examples are provided to aid those skilled in the art in practicing the present disclosure.

### Examples

### Phage Display Library Screen

A human B cell-derived scFv phage display library (Creative BioLabs, USA) was used to pan against the recombinant human EphA10 extracellular domain protein (Glu34-Ala565), transcript variant 3, fused with DDK/His tag at C-terminal (Creative Biomart, USA, Catalog no. EPHA10-369H). Positive clones were screened using ELISA. A unique CMU #13 were identified by DNA sequencing. The binding specificity of the unique clones was confirmed by FACS using full-length human EphA 10-expressing NIH 3T3 cells.

### Affinity Assay (ELISA)

ScFv sequences were fused with mouse IgG2a sequence and sub-cloned into pcDNA3.1 plasmid. Three ScFv-mouse IgG2a plasmids were transfected into HEK293 cells. Three ScFv-mouse IgG2a antibodies were purified by protein A resin. Three antibodies were tested for indirect ELISA against coated 9 EPHA antigens (EphA1-8 and 10, R&D systems).

The results are shown in FIG. 1. CMU #13 is shown to only bind to EphA10 without binding to other EphAs.

CMU #13 was also subjected to a Biacore assay. The result is shown in Table 2. CMU #13 with a KD value of 1.79×10⁻⁹ M against EphA 10 showing high affinity.

TABLE 2
Affinity constant

| Kₐ(M⁻¹ × S⁻¹) | K_{d}(S⁻¹) | K_{D}(M) | Chi² (RU²) |
|---|---|---|---|
| 1.61 × 10⁶ | 2.88 × 10⁻³ | 1.79 × 10⁻⁹ | 9.92 × 10⁻¹ |

The sample was run in triplicates.

K_{D} values (affinity constant or dissociation equilibrium constant) were calculated from all the binding curves based on their global fit to 1:1 binding model by Biacore 8k data analysis softward.

Chi² is the average of squared residuals. (Rmax=55.6 RU)

### Construction of EphA10-CAR-T Lentiviral Vector

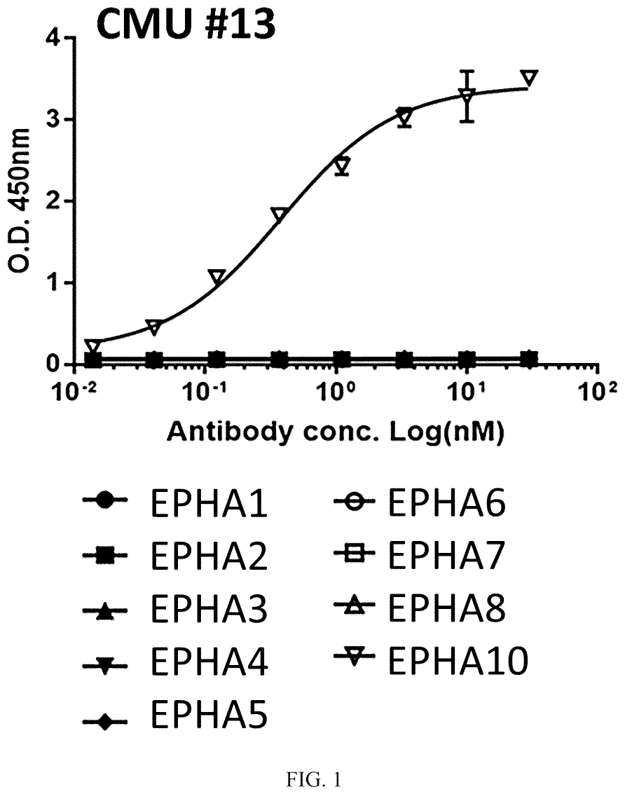

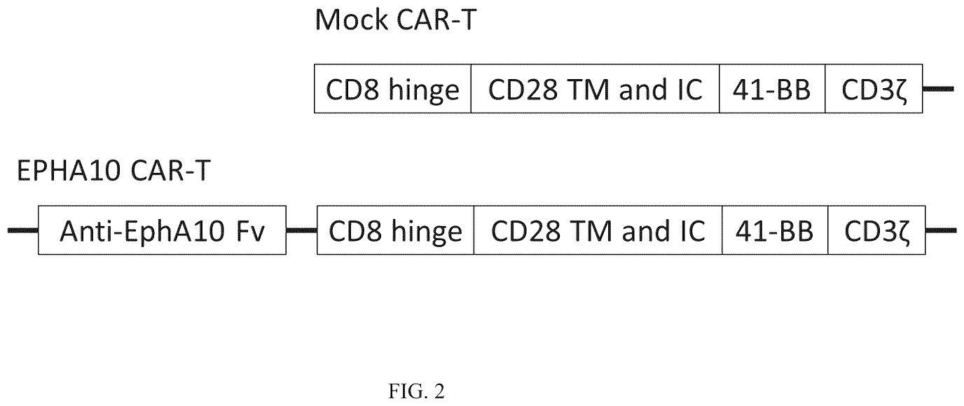

CMU #13 ScFv (SEQ ID NO: 9) was cloned and inserted to a third-generation CAR-T lentiviral vector containing CD8 hinge, CD28 TM and IC, 41-BB and CD33 (as shown in FIG. 2). The sequence of CMU #13 ScFv was synthesized and sub-cloned into a third-generation CAR-T lentiviral vector (pCDH-EF1a-MCS) (Creative BioLabs, USA). To generate EphA10 CAR lentivirus, the third-generation CAR-T lentiviral vector and packaging vectors (LentiArt™ Virus Packaging Kit, Creative Biolabs, USA, Cat. No. CART-027CL) were co-transfected into HEK293 cells.

Generation of EphA10-CAR iPSC (EphA10-CAR iPSC)

Human iPSCs purchased from SBI System Biosciences were used for the generation of EphA10-CAR iPSCs. Briefly, iPSCs were stably transduced with EphA10 CAR lentivirus encoding a third generation CAR structure of CMU #13 ScFv (SEQ ID NO: 9). The EphA10-CAR iPSC lines were established after sorting for high expression of the fluorescent marker. All EphA10-CAR iPSC lines were maintained in culture on Geltrex-coated cultureware with Pluto medium and passaged every 5-7 days. EphA10-CAR iPSC lines were tested for mycoplasma contamination every 2 months.

Characterization and Assessment of Pluripotency of EphA10-CAR iPSCs

For assessment of expression of endogenous pluripotency genes, total mRNA from EphA10-CAR iPSC was extracted with Trizol. Reverse transcription was performed with Superscript III kit and qRT-PCR was performed with SYBR green system. Reactions were carried out in duplicate in an ABI PRISM 7500 Sequence Detection System. Expression was calculated by relative quantification using the 2^{-ΔΔCt} method with GAPDH as endogenous control.

For flow cytometric analysis, EphA10-CAR iPSCs were stained with the following fluorophore-conjugated antibodies: SSEA-4-AlexaFluor647, Tra-1-81-AlexaFluor647, and Tra-1-60-AlexaFluor647. All flow cytometry analysis was done on a LSRII cytometer and analyzed using FlowJo software, Ver. 9.5.2.

For teratoma formation assays, undifferentiated EphA10-CAR iPSCs were suspended in Matrigel. Approximately 2×10⁶ cells were injected subcutaneously into 6- to 12-week-old female NOD-SCID IL2Rycnull mice. Five to six weeks later, teratomas were surgically dissected and fixed in 4% formaldehyde. Paraffin-embedded samples were stained with hematoxylin and eosin for histological analysis.

For karyotyping, standard G-banding analysis was done at the MSKCC molecular cytogenetics core facility. Chromosome analysis was done on a minimum of 12 4,6-diamidino-2-phenylindole (DAPI)-banded metaphases.

T Cells Differentiation from EphA10-CAR iPSCs and Expansion of EphA10-CAR-T Cells

EphA10-CAR iPSCs were differentiated to CD8αβ double-positive (DP) cells using the OP9 and OP9/DLL1 stromal cell coculture systems. In brief, iPSC colonies were dissociated using trypsin (0.25%) and collagenase IV (1 mg/mL) and mechanically disrupted into small clumps by pipetting. About 600 iPSC clumps were collected and plated on gelatin precoated OP9 dishes filled with OP9 medium, that is, α-MEM (Invitrogen) with 20% FCS. On day 13, colonies were treated with collagenase Type IV (50 U/mL) and trypsin-EDTA (0.05%). Cells were plated in a OP9/DLL1 semiconfluent dish on OP9 medium containing hIL-7 (5 ng/ml), hFlt-3L (5 ng/ml), and hSCF (5 ng/mL). On day 15, semiadherent cells were collected and passage into a new dish layered with OP9/DLL1 cells. From this point, passage was done every 7 days. On day 40, floating cells were collected and CD4/8 DP cells were enriched by using CD4 microbeads (Milteny Biotec). CD4/8 DP cells were stimulated with anti-human CD3 Ab (500 ng/ml; OKT-3, eBioscience), anti-human CD28 Ab (2 g/mL; eBioscience), 100 IU human interleukin 2 (hIL-2) and 5 ng/mL hIL-7. On day 4-7 after stimulation, CD8αβ double-positive (DP) cells can be collected for molecular analysis.

### Generation of EphA10-CAR T (EphA10-CAR T)

Human CD8⁺ T cells were used for the generation of EphA10-CAR T. Briefly, CD8⁺ T cells were stably transduced with CAR lentivirus encoding a CMU #13 ScFv (SEQ ID NO: 9). All EphA10-CAR T cell lines were maintained in KBM502 medium (Kohjin Bio, Japan). EphA10-CAR T cell lines were tested for mycoplasma contamination every 2 months.

### Flow Cytometry

Following monoclonal antibodies were used; anti-CD3, anti-CD4, anti-CD8, anti-CD34, and anti-CD56 mAbs. All mAbs were purchased from BD Biosciences or eBioscience. For the detection of T cells expressing ScFv specific EphA10, fluorescent-conjugated EphA10 peptide was used. The expression of CD69 (activation marker) will be examined for the activation of CAR-T cells. Flow cytometry was performed using Attune NxT flow cytometer (Thermo Fisher) with FlowJo software (TreeStar).

### Cytotoxicity Assays

Cytotoxic potential of EphA10-CAR-T cells was evaluated in non-radioactive cytotoxicity assay. The EphA10-CAR-T cells were co-cultured with EphA10-positive MDA-MB-231 cells at a effector to target ratio of 10 (E/T ratio=10; effector cell: EphA10-CAR-T cell; target cell: MDA-MB-231 cell) for 24 hours. The cell viability of MDA-MB-231 was assayed with MTT assay.

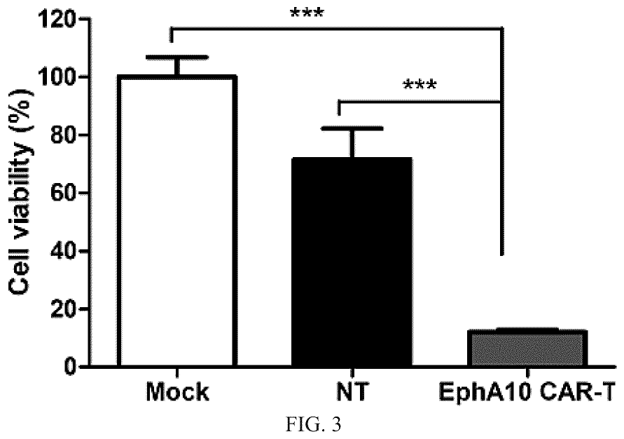

The results are shown in FIG. 3. The treatment of EphA10-CAR-T cells significantly reduced the viability of MDA-MB-231.

### In Vivo Tumor Model

6- to 12-week-old female NOD-SCID IL2Rycnull mice were inoculated with 10⁶ human cancer cells (day 0), MDA-MB-231 cells expressing a firefly luciferase protein (Luc). EphA10-CAR-T cells or T cells were injected i.v. along with IL-2 (50,000 U/mouse) at day 7. T-cell dose was based on the percentage of CAR⁺ cells as measured by pre-injection flow cytometric analysis. IL-2 administration was continued twice a week. Tumor burden was monitored twice per week by in vivo bioluminescence imaging (IVIS 100 Imaging System). Living Image software Version 4.3.1 was used to acquire and quantify the bioluminescence imaging data sets.

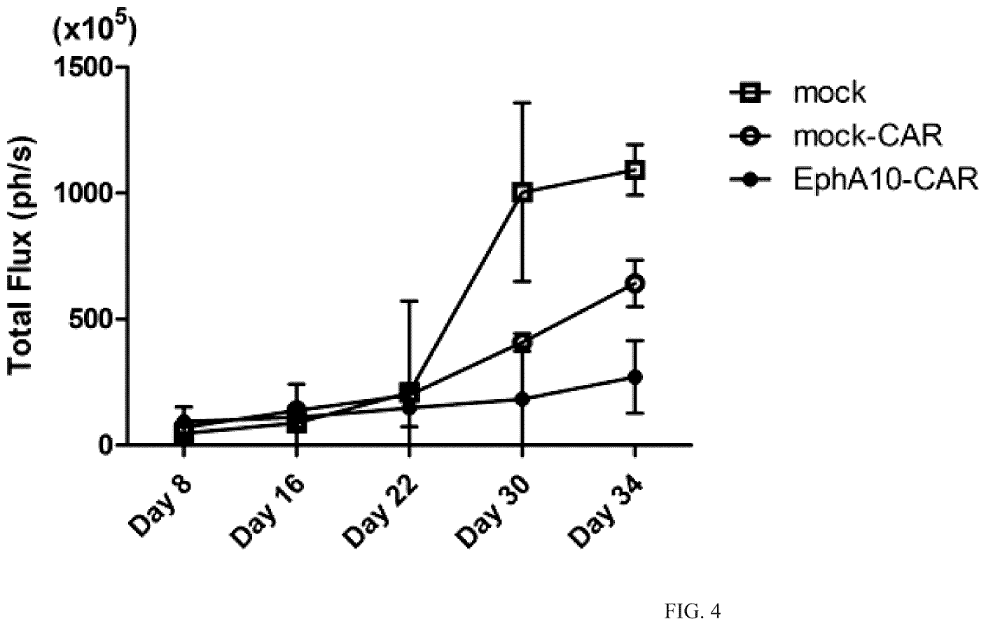

In the in vivo tumor model, the tumor inhibition effect of anti-EphA10-CAR-T cell was determined with using a breast cancer xenograft animal model. The anti-EphA10-CAR-T cells (1×10⁷ cells/injection) was injected via femoral vein on day 8, 15 and 22, respectively. The results show that the luminescence signal of the tumor in the anti-EphA10-CAR-T cell group started to decrease after the second CAR-T cell injection, which indicates that anti-EphA10-CAR-T cells inhibited tumor proliferation more than Mock-CAR-T cells, compared with the Mock-CAR-T cell treatment group (FIG. 4).

### Anti-EphA10 CAR-T Cell Triggers Massive Apoptosis in Tumors

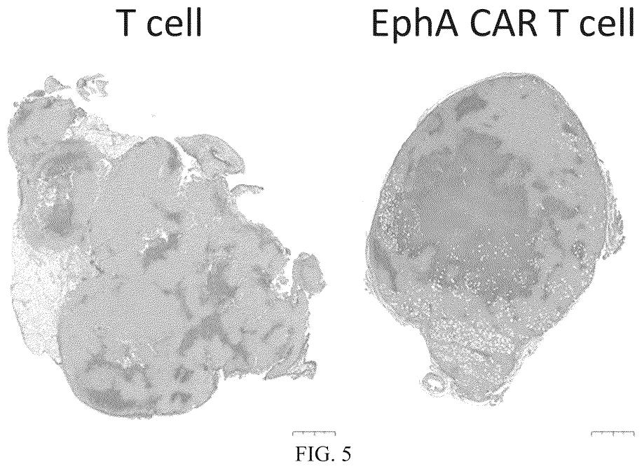

We found that anti-EphA10 CAR-T cells induced extensive apoptosis in tumor tissues (FIG. 5). In sectioning and TUNEL assay analysis, the results showed that anti-EphA10 CAR-T cells exerted effective anti-tumor ability and triggered apoptosis of human tumors in the animal model.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

What is claimed is:

1. A polypeptide comprising an antibody or antigen-binding fragment thereof that is specific to an epitope in ephrin type-A receptor 10 A10 (EphA10); wherein the antibody or antigen-binding fragment thereof comprises complementarity determining regions (CDRs) of a heavy chain variable region and complementarity determining regions of a light chain variable region, wherein the complementarity determining regions of the heavy chain variable region comprise CDRH1, CDRH2 and CDRH3 regions, and the complementarity determining regions of the light chain variable region comprise CDRL1, CDRL2 and CDRL3 regions, and wherein:
the CDRH1 region comprises the amino acid sequence of SEQ ID NO: 1 or a substantially similar sequence thereof; the CDRH2 region comprises the amino acid sequence of SEQ ID NO: 2 or a substantially similar sequence thereof; the CDRH3 region comprises the amino acid sequence of SEQ ID NO: 3 or a substantially similar sequence thereof; and
the CDRL1 region comprises the amino acid sequence of SEQ ID NO: 4 or a substantially similar sequence thereof; the CDRL2 region comprises the amino acid sequence of SEQ ID NO: 5 or a substantially similar sequence thereof; the CDRL3 region comprises the amino acid sequence of SEQ ID NO: 6 or a substantially similar sequence thereof.

2. The polypeptide according to claim 1, wherein the antibody or antigen-binding fragment thereof comprises a heavy chain variable region comprising the amino acid sequence of SEQ ID NO: 7 or a substantially similar sequence thereof; and a light chain variable region comprising the amino acid sequence of SEQ ID NO: 8 or a substantially similar sequence thereof.

3. The polypeptide according to claim 1, wherein the antibody is a monoclonal antibody, chimeric antibody, humanized antibody, or human antibody.

4. The polypeptide according to claim 1, wherein the antibody is multi-specific.

5. The polypeptide according to claim 1, which is conjugated with a therapeutic agent.

6. The polypeptide according to claim 5, wherein the therapeutic agent is selected from the group consisting of antimetabolites, alkylating agents, alkylating-like agents, DNA minor groove alkylating agents, anthracyclines, antibiotics, calicheamicins, antimitotic agents, topoisomerase inhibitors, HDAC inhibitor, proteasome inhibitors, and radioisotopes.

7. The polypeptide according to claim 1, which is expressed on a surface of a cell.

8. The polypeptide according to claim 7, wherein the cell is an immune cell or a stem cell.

9. (canceled)

10. (canceled)

11. The polypeptide according to claim 1, comprising the antigen binding portion comprising the antibody or antigen-binding fragment thereof; and
at least one costimulatory signaling domain selected from CD27, CD28, 4-1BB, OX40, CD30, CD40, ICOS, ICAM-1, LFA-1, CD2, CD7, LIGHT, NKG2C, B7-H3, a ligand that specifically binds with CD83, CDS, GITR, BAFFR, HVEM, SLAMf7, NKP80, CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, C49f, ITGAD, CD11d, ITGAE, CD103 ITGAL, CD11a, LFA-1, ITGAM, CD11b, ITGAX, CD11c, ITGB1, CD29, ITGB2, CD18, ITGB7, TNFR2, TRANCE/RANKL, DNAM1, SLAMF4, CD84, CD96, CEACAM1, CRTAM, Ly9, PSGL1, C100, CD69, SLAMF6, SLAM, BLAME, SELPLG, LTBR, LAT, GADS, PAG/Cbp, SLP-76, NKp44, NKp30, or NKp46.

12. (canceled)

13. The polypeptide according to claim 11, which further comprises a primary signaling domain or a hinge domain.

14. (canceled)

15. The polypeptide according to claim 11, which comprises the antigen binding portion, a CD8 hinge, CD28, 41-BB and CD3ζ.

16. (canceled)

17. (canceled)

18. (canceled)

19. (canceled)

20. A vector encoding the polypeptide according to claim 1.

21. (canceled)

22. A genetically engineered cell expressing the polypeptide according to claim 1.

23. The genetically engineered cell or the cell differentiated therefrom according to claim 22, which is an immune cell or a stem cell.

24. (canceled)

25. (canceled)

26. (canceled)

27. (canceled)

28. (canceled)

29. (canceled)

30. (canceled)

31. (canceled)

32. (canceled)

33. A method for inhibiting EphA10-mediated signaling in a subject in need, comprising administering an effective amount of the polypeptide according to claim 1 to the subject.

34. A method for treating, prophylactic treating and/or preventing diseases and/or disorders caused by or related to EphA10 activity and/or signaling in a subject afflicted with the diseases and/or disorders, comprising administering an effective amount of the polypeptide according to claim 1 to the subject.

35. (canceled)

36. The method according to claim 34, wherein the diseases and/or disorders are a tumor.

37. The method according to claim 36, wherein the tumor is selected from the group consisting of renal cell carcinoma, pancreatic carcinoma, breast cancer, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, and melanoma.

38. A method for detecting EphA10 in a sample comprising contacting the sample with the polypeptide according to claim 1.

39. A method for neutralizing EphA10 in a subject in need, comprising administering an effective amount of the polypeptide according to claim 1 to the subject.

40. (canceled)


SEQUENCE LISTING (10 sequences)

SEQ ID NO: 1 - protein, 5 aa, synthetic construct
SYAIS

SEQ ID NO: 2 - protein, 17 aa, synthetic construct
GIIPIFGTANYAQKFQG

SEQ ID NO: 3 - protein, 13 aa, synthetic construct
EGDSYGYYYGMDV

SEQ ID NO: 4 - protein, 13 aa, synthetic construct
SGSSSNIGSNYVY

SEQ ID NO: 5 - protein, 7 aa, synthetic construct
ENYLRPS

SEQ ID NO: 6 - protein, 11 aa, synthetic construct
AAWDDSLNGYV

SEQ ID NO: 7 - protein, 122 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREGDSYGYYYGMDVWGQGTTVTVSS

SEQ ID NO: 8 - protein, 111 aa, synthetic construct
QPVLTQPPSASGTPGQRVTISCSGSSSNIGSNYVYWYQQLPGTAPKLLIYENYLRPSGVPDRFSGSKTGTSASLAISGLQSEDEADYYCAAWDDSLNGYVFGTGTQLTVLS

SEQ ID NO: 9 - protein, 248 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREGDSYGYYYGMDVWGQGTTVTVSSGGGGSGGGGSGGGGSQPVLTQPPSASGTPGQRVTISCSGSSSNIGSNYVYWYQQLPGTAPKLLIYENYLRPSGVPDRFSGSKTGTSASLAISGLQSEDEADYYCAAWDDSLNGYVFGTGTQLTVLS

SEQ ID NO: 10 - DNA, 744 bp, synthetic construct
  source 1..744: mol_type = other DNA
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagaaggggacagctatggttactactacggtatggacgtctgggggcaggggaccacggtcaccgtctcgagtggtggaggcggttcaggcggaggtggttctggcggtggcggatcgcagcctgtgctgactcagcccccctcagcgtctgggacccccgggcagagggtcaccatctcttgttctggaagcagctccaacatcggaagtaattatgtatactggtaccagcagctcccaggaacggctcccaaactcctcatctatgagaattatctgcggccctcaggggtccctgaccgattctctggctccaagactggcacctcagcctccctggccatcagtgggctccagtctgaggatgaggctgattattactgtgcagcatgggatgacagcctgaatggttatgtcttcggaactgggacccagctcaccgttttaagt